(19) United States
(12) Patent Application Publication
(10) Pub. No.: US 2010/0112048 A1
(43) Pub. Date: May 6, 2010

(54) Dosage Regimen and Medicament For Reducing Risk of Onset or Progression of Dementia by Administration of Specific Vitamins and Nsaid

(76) Inventor: Majid Fotuhi, Cockeysville, MD (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/161,734

(22) PCT Filed: Jul. 19, 2006
(86) PCT No.: PCT/US2006/028025

§ 371 Date: Jan. 25, 2010

Related U.S. Application Data
(60) Provisional application No. 60/761,344, filed on Jan. 24, 2006.
(60) Provisional application No. 60/831,218, filed on Jul. 17, 2006.

(51) Int. Cl. A61K 31/355; A61K 31/60; A61K 31/426; A61K 31/44; A61K 31/381; A61K 31/4035; A61K 31/423; A61K 31/4015; A61K 31/405; A61K 31/4152; A61K 31/53; A61K 31/5415; A61K 9/28; A61P 25/28; A61K 9/48

(52) U.S. Cl. 424/463; 514/161; 514/458; 514/365; 514/352; 514/448; 514/416; 514/375; 514/423; 514/420; 514/404; 514/243; 514/226.5; 424/474

(57) ABSTRACT

The present invention relates to a synergistic combination for preventing the onset and/or progression of dementia or Alzheimer's disease in individuals at increased risk thereof for example because of family history, genetic factors, and/or environmental factors. This combination comprises synergistically effective amounts of vitamin C, vitamin E, DHA and at least one NSAID such as ibuprofen.

### RELATED APPLICATIONS

This PCT patent application claims benefit of priority to provisional application 60/761,344 entitled “Preventing Dementia” filed on Jan. 24, 2006 and provisional application filed Jul. 17, 2006 (number not yet assigned) having the same title as the present application and both filed in the name of Majid Fotuhi which applications are incorporated by reference in their entireties herein.

### BACKGROUND OF THE INVENTION

With the current increase in the numbers and proportion of the elderly in the population, cognitive decline and dementia are major public health issues. Alzheimer's disease, the most common form of dementia, already challenges our health care system in the United States and Canada, and the associated costs are projected to skyrocket in the coming decades. Disabling memory loss is not limited to Western countries. Roughly, 24.3 million people globally have dementia, and 4.6 million cases arise every year. There is a new case of dementia arising every seven seconds. Given these numbers, there is an urgent need to find interventions that can effectively slow or even prevent the onset of dementia and the rate of cognitive decline associated with aging.

Currently, the underlying causes of dementia are not entirely understood. It is known that inflammation and the production of reactive oxygen apparently play a major role in the neuronal injury and brain atrophy that occur during aging and dementia. It is also known that during the early stages of Alzheimer's disease that a specific protein (amyloid) accumulates in the brain and forms gum-like insoluble plaques (amyloid plaques). These plaques are known to damage brain cells directly and indirectly by triggering inflammatory reactions, which in turn cause more brain damage.

Inflammatory markers are elevated in patients exhibiting dementia. However, notwithstanding the limited understanding of the in vivo mechanisms that occur during dementia, and a great deal of interest from government agencies, research universities and the pharmaceutical industry, to date there is no medication available for preventing or delaying the dementia and memory loss that occurs with aging or dementia. There is some anecdotal evidence that mental health is better maintained in individuals who conduct daily activities that are stimulating to the brain. For example an autopsy study of the brains of nuns who were very mentally active until death (many of whom had lived until their late 80's and 90's) revealed that a substantial number of these autopsied brains appeared to exhibit significant anatomical signs of plaques and disease. Notwithstanding, these individuals did not display any clinical symptoms of dementia or Alzheimer's at the time of their death. Based on this and other anecdotal evidence, the Alzheimer's Association recently began a publicity campaign to promote healthy eating, exercise, and brain stimulation in order to maintain brain function during aging. For example, they have promoted elderly people to conduct daily mental exercises such as completing mazes or crossword puzzles or playing computer games to maintain mental agility. However, they have not yet offered any specific means or medicament for preventing dementia and Alzheimer's disease.

Several new medications reportedly are helpful in reducing the severity and frequency of symptoms in patients with Alzheimer's disease. These include Aricept, Razadyne, Exelon, and Namenda. The first two of these medications have been studied in patients with marked memory loss at risk for developing Alzheimer's disease. Neither of these drugs were shown to significantly prevent the onset of Alzheimer's disease. This is not surprising since neither drug targets the source of brain damage that leads to dementia.

As noted above, serum inflammatory markers are elevated in patients with dementia. Some observational studies have shown that individuals taking non-steroidal anti-inflammatory drugs (NSAID's) such as ibuprofen exhibit a lower incidence of Alzheimer's disease. Other studies have shown improved cognition in elderly patients taking the anti-oxidant vitamins E and C. Vitamin E levels are sometimes low in patients with cognitive impairment or dementia and treatment thereof with vitamin E can even delay transfer to a nursing home. Another potent anti-oxidant, DHA (docosahexanoic acid) has also been reported to help reduce the damage caused by the plaques and tangles of Alzheimer's disease in the brain. These studies favor use of NSAID's and/or anti-oxidant vitamins for preventing dementia.

Because the reported research into the cause of brain damage in Alzheimer's disease points to inflamation, anti-inflammatory medications have been suggested as a preventative. For example, patients who suffer from rheumatoid arthritis and take anti-inflammatory drugs on a daily basis have a reduced incidence of Alzheimer's disease onset. Other studies, including one made at Johns Hopkins, revealed that participants taking vitamins E and C were less likely to become demented. However, to the best of the inventors' knowledge no study has suggested the combined administration of NSAID's and anti-oxidant vitamins.

### BRIEF DESCRIPTION OF THE FIGURES

FIG. 1 contains estimated trajectories of 3MS scores over 3 Waves of observation spanning 8 years of follow-up for the full sample.

FIG. 2 contains estimated trajectories of 3MS scores over 8 years of follow-up in individuals with no APOE epsilon 4 alleles.

FIG. 3 contains estimated trajectories over 3 Waves of observation spanning 8 years of follow-up among those with one or more APOE epsilon 4 alleles.

### BRIEF DESCRIPTION OF THE INVENTION

The present invention provides a novel synergistic combination for preventing or delaying the onset of dementia, chronic intellectual decline (CID) and/or Alzheimer's disease, especially in individuals at high risk because of heredity, genotype, and/or environmental factors.

More specifically, the present invention relates to the prevention and/or delaying of the onset or progression of dementia, OD and/or Alzheimer's disease in individuals at increased risk by administering the combination of anti-oxidant vitamins (vitamin E and vitamin C), docosahexanoic acid (DHA) and at least one NSAID, preferably ibuprofen.

The present invention further relates to a novel dosage regimen for preventing or delaying the onset of dementia, CID and/or Alzheimer's disease in individuals by administering daily vitamin C, vitamin E, DHA, and an NSAID, preferably ibuprofen, or pharmaceutically acceptable derivatives or salts of said active constituents.

The present invention further relates to a medicament for preventing or delaying the onset of dementia, CID and/or Alzheimer's disease comprising synergistically effective amounts of vitamin E, vitamin C, DHA, and at least one NSAID, preferably ibuprofen, or pharmaceutically acceptable derivatives or salts thereof. These medicaments will preferably comprise tablets or capsules suitable for once or twice daily administration.

### DETAILED DESCRIPTION OF THE PRESENT INVENTION

As discussed supra, some anecdotal studies have suggested the use of anti-oxidant vitamins as well as the use of NSAID's such as ibuprofen for preventing or delaying the onset of dementia or for treating dementia. However, a medicament or nutrient supplement or combination regimen for preventing dementia CID and/or Alzheimer's disease comprising the combination of anti-oxidant vitamins (vitamin C and Vitamin E), the anti-oxidant DHA and an NSAID (ibuprofen) was previously not known. Also, the synergistic benefits of such a combination with respect to the prevention or delay in the onset of dementia, CID and/or Alzheimer's disease in patients, especially those at elevated risk of developing dementia, CID and/or Alzheimer's disease were previously unknown.

Thus, the present invention provides a novel dosage regimen and medicament or nutrient supplement for use therein for preventing and/or delaying the onset or progression of dementia, CID and/or Alzheimer's disease in individuals at elevated risk for developing dementia or CID or individuals exhibiting the early signs of dementia or CID. Such individuals include for example persons with a family history of dementia or Alzheimer's disease, and individuals who comprise genetic markers that correlate to a higher risk of developing dementia or Alzheimer's disease such as APOE-epsilon-4 carriers.

In particular the subject invention provides novel medicaments or nutrient compositions comprising synergistically effective amounts of vitamin C, vitamin E, DHA, and at least one NSAID (non-steroidal anti-inflammatory drug). These NSAIDs include by way of example salicyclic acid (aspirin), acetyl salicyclic acid, diflunisal, choline magnesium trisalicylate, salicylate, benorylate, flufenamic acid, mefenamic acid, meclofenamic acid, niflumic acid, diclofenac, fenclofenac, aclofenac, fentiazac, ibuprofen, flurbiprofen, ketoprofen, naproxen, fenoprofen, fenbufen, suprofen, indoprofen, tiaprofenic acid, benoxaprofen, piroprofen, tolmetin, zomepirac, clopinac, indomethacin, suldinac, phenylbutazone, oxyphenbutazone, azapropazone, feprazone, piroxicam, isoxicam, nabumetone, and sudoxicam.

Typically the NSAID will comprise ibuprofen, suldinac, meclofenamic acid, aspirin, diclofenac sodium, naproxen, flurbiprofen, or nabumetone and most typically ibuprofen.

“Synergistically effective amounts” herein refers to an amount of these constituents that exhibits a statistically synergistically effective effect with respect to preventing and/or delaying the onset or progression of dementia in treated individuals relative to the individual constituents. As discussed infra, there is statistical evidence in individuals at risk of developing dementia, such as APOE epsilon-4 carriers that these constituents when administered in combination as described herein exhibit a synergistic effect on preventing dementia which was previously unknown and unanticipated. This synergy was evidenced by a significantly, reduced decline in mental scores in the Modified Mini-Mental State Exam scores. These individuals after adjusting for age, gender, APOE genotype, hypertension, cholesterol, stroke, coronary artery bypass surgery, and MI, performed better than individuals not receiving the combination by approximately 1 point every 3 years (P less than 0.05). This enhancement was more pronounced in individuals with one or more APOE epsilon 4 alleles who increased 2.19 points every 3 years (P less than 0.05)

As explained in greater detail infra, it is believed that this synergistic efficacy is achieved because of the combined anti-inflammatory effect of the NSAID which reduces the amount of amyloid in the brain, the substrate that causes inflammation and the anti-inflammatory effects of the anti-oxidant vitamins. Thereby the invention is believed to provide a synergistic “2-pronged attack” on the factors and cascade of events that results in dementia, CID and/or Alzheimer's disease pathology.

The subject synergistic combination may be administered as a single combined medicament or nutritive supplement, or separately. Administering these constituents in combination is preferred because this simplifies patient compliance.

These constituents may be in various dosage forms including oral and transdermal dosage forms. Preferably the medicament will be administered in the form of a tablet, capsule, powder or liquid dosage form. For example, the medicament may be comprised in straws using the proprietary “Dose Sipping Technology” to facilitate usage in elderly patients who may have difficulty swallowing tablets. Alternatively the medicament may be administered in the form of a transdermal patch that provides for controlled delivery of the constituents therein. Still alternatively the medicament may be in the form of once-daily or less preferably twice-daily administrable tablets or capsules.

If administered as a tablet the medicament may further comprise one or more functional or non-functional coatings, such as enteric coatings. The use of such coatings may help reduce stomach toxicity, such as gastric ulcers since some NSAIDs such as aspirin and ibuprofen are associated therewith especially at high dosages.

Typically the medicament will comprise not more than 3000 mg of vitamin C, not more than 1000 iu of vitamin E, not more than 3000 mg of DHA, and an amount of a NSAID which is effective but does not elicit undue toxicity. For example in the case of ibuprofen not more than 300 mg, suldinac not more than 300 mg, meclofenamic acid not more than 150 mg, aspirin not more than 150 mg, diclofenac sodium not more than 150 mg, naproxen not more than 500 mg, flurbiprofen not more than 300 mg and nabumetone not more than 1000 mg. In the present invention in all instances including the claims when dosage amounts are recited, and specific numbers, in all instances it should be understood that these ranges and numbers and dosages are approximations, and that the numbers, ranges and dosages can be understood as if the modifier “about” were present.

For example, a medicament according to the invention may comprise from 100-2000 mg of vitamin C, from 50-600 iu of vitamin E, from 100-2000 mg of DHA, and at least one NSAID wherein if ibuprofen ranges from 20-200 mg, if suldinac from 10-200 mg, if meclofenamic acid from 5-100 mg, if aspirin from 10-100 mg, if diclofenac sodium from 5-100 mg, if naproxen from 20-200 mg, if flurbiprofen from 20-200 mg, and if nabumetone from 50-500 mg.

In another more specific example, the medicament may comprise from 20-1000 mg of vitamin C, from 100-400 iu of vitamin E, from 200-80 mg of DHA and an amount of at least one NSAID which ranges from 30-100 mg for ibuprofen, from 30-100 mg for suldinac, from 10-40 mg for meclofenamic acid, from 20-50 mg for aspirin, from 10-40 mg for diclofenac sodium, from 50-150 mg for naproxen, from 30-100 mg for flurbiprofen, and from 100-300 mg for nabumetone.

In an even more specific embodiment the medicament may comprise 500 mg of vitamin C, 200 iu of vitamin E, 500 mg of DHA, and an mount of at least one NSAID which is 50 mg if ibuprofen, 50 mg if suldinac, 20 mg if meclofenamic acid, 30 mg if aspirin, 20 mg if diclofenac sodium, 80 mg if naproxen, 50 mg if flurbiprofen and 150 mg if nabumetone.

The constituents contained in the subject medicaments or nutrient supplements may comprise their known isomeric forms, racemates, pharmaceutically acceptable salts, and active derivatives thereof including prodrug forms. Also, the subject medicaments or nutrient supplements may comprise other active and non-active constituents and excipients such as flavor enhancers, tabletting agents, surfactants, emulsifiers, and the like.

The subject medicaments or nutrient supplements are administered to individuals at increased risk of developing dementia or Alzheimer's disease because of factors such as age, family history of dementia, early memory loss or Alzheimer's disease, the presence or absence of genetic markers that correlate to an increased incidence of dementia, memory loss or Alzheimer's disease, and environmental factors that correlate to an increased incidence of dementia, memory loss or Alzheimer's disease. For example it has been suggested that ingestion of some metal such as aluminum may correlate to an increased incidence of Alzheimer's disease.

Typically, the treated individual will be at least 55 years, more typically at least 65 years, and even more typically 70 years or older. Especially preferred individuals for use of the subject medicaments or nutrient supplements are APOE-epsilon-4 carriers. Other preferred individuals may comprise persons at risk of developing stroke related dementia, and individuals with several close family members with Alzheimer's disease or early memory loss or dementia. Also, the subject medicaments or nutrient supplements may be administered to individuals with early signs of the onset of dementia or memory loss such as increased forgetfulness.

The subject medicaments or nutrient supplements are preferably administered as once-daily or twice-daily dosage regimens to subjects or individuals at increased risk of dementia, early memory loss, and/or Alzheimer's disease.

As noted above, several previous studies have alleged obtained improved cognition and/or prevention of dementia with the use of either anti-oxidant vitamins E and C or NSAIDs. However, the effect of their combined use on the onset of dementia has not yet been studied.

Therefore, it was the objective of the present inventor to examine whether taking vitamins E and C in combination with an NSAID would potentially slow the rate of cognitive decline in the elderly and others at elevated risk of developing dementia and also to determine whether the combination elicits a synergistic benefit in preventing or slowing dementia or CID relative to the individual constituents in the combination.

Experimental Study Design Parameters

The present inventor conceived the idea that a medicament comprising an NSAID, vitamin E and Vitamin C as well as DHA in appropriate amounts would elicit synergistic effects with respect to retarding or slowing the onset of dementia in individuals at risk, e.g. those with genetic predisposition to Alzheimer's disease. such as APOE epsilon-4 carriers. Therefore, the inventor conceived the idea of a once-a-day or twice-daily medicament or nutritional supplement containing synergistic amounts of DHA, vitamin E and Vitamin C, and an NSAID such as ibuprofen for preventing or slowing the onset of dementia in persons at risk such as the elderly and those with genetic or environmental risk factors. As part of this conception the present inventor, who was aware of an ongoing longitudinal study of elderly individuals in Utah, including some individuals ingesting and those not ingesting vitamins and/or NSAIDs, further conceived the idea of assessing in these subjects whether the proposed combination indeed had any preventative or retarding effect on the onset of dementia and also whether the proposed combination elicited synergistic effects. These results are discussed below. As the inventor had hoped and anticipated the subject combination was found to elicit a statistically demonstrable effect on dementia particularly in individuals that were genetically predisposed.

Design, Setting, and Participants: Prospective study, conducted over eight years among 3376 non-demented elderly, aged 65 years or older, living in their community in Cache County, Utah. Random effects models examined the association between separate and combined consumption of vitamin E, vitamin C, and NSAIDs with cognitive performance at baseline (1995-1997), at wave II (1998-2000), and at wave III (2003-2004).

Main Outcome Measure: Longitudinal evaluation with a standardized test of cognitive performance, the Modified Mini-Mental State Examination (3MS), with scores ranging from 0-100.

Results: As described in detail infra, a total of 85 participants (2.5%) reported taking vitamins E and C in combination with NSAIDs (“super-users” group) at baseline. They performed significantly better than non-users in 3MS over time, about 1.0 points every three years (p<0.05), after adjustments for age, sex, hypertension, high cholesterol, stroke, coronary artery bypass graft surgery, or myocardial infarction. This association was particularly evident in those with one or more APOE-e4 alleles, at 2.19 points every three years (p<0.05). Among those with no APOE epsilon 4 alleles, only vitamin E and C users (4.5%) performed better than non-users in the 3MS baseline by 1.62 points (P less than 0.05), however, they declined over time at the same rate as non-users. There was no increase in mortality rate in any of the vitamins and/or NSAID groups.

Conclusion: Individuals at risk of dementia or Alzheimer's disease, such as the elderly taking the combination of the anti-oxidant vitamins E and C in combination with NSAIDs appear to have a slower decline in their cognitive performance over time. Interestingly, this synergistic benefit appears more significant and that greater synergistic results are observed in individuals with one or more APOE-e4 alleles.

Inventor's Rationale:

With the rapid rise in the proportion of elderly in the population and the increasing number of patients with Alzheimer's disease every year, there is an urgent need to find effective ways that can even modestly slow the rate of cognitive decline with aging (1,2). The main current theory for the cause of this disease focuses on the production and aggregation of amyloid protein into insoluble plaques and aggregation of phosphorylated tau protein into tangles (1-3). The subsequent inflammatory reactions including the production of reactive oxygen species are believed to contribute to the subsequent neuronal injury that leads to synaptic loss and brain atrophy (4-7). Markers of protein oxidation and levels of inflammatory proteins are higher in elderly with cognitive decline (8-10).

Growing evidence suggests a potential role of anti-oxidant vitamins or Non-Steroidal Anti-Inflammatory Drugs (NSAIDs) for primary prevention of dementia (2,4-5, 10-19). The analysis of data from the Cache County Study, a longitudinal investigation of over 5000 community-dwelling participants since 1995, also indicated a reduced risk of Alzheimer's disease in elderly taking anti-oxidant vitamins or NSAIDs. (18,19) However, some other studies have not noted significant effect for prevention or treatment of Alzheimer's disease with either anti-oxidant vitamins or NSAIDs by themselves The exact explanation of the apparent discrepancy remains elusive. Anti-oxidant vitamins and NSAIDs may each be necessary, but not sufficient, for slowing the brain pathology that leads to Alzheimer's disease. Possible additive or synergistic benefit for their combined use in preventing or slowing dementia or CID has not yet been studied.

Since these molecules target different steps in the pathogenesis of Alzheimer's disease, it was decided to test the hypothesis that their combined use may have a synergistic benefit in slowing the rate of cognitive decline with aging. In the study, an adapted Version of a standard test of cognitive performance, the Modified Mini-Mental State Exam (3MS), was used to monitor study participants at different times

The apolipoprotein E (APOE) genotype strongly predicts the onset, severity, and rate of progression in Alzheimer's disease (20,21). Individuals with one copy of APOE epsilon 4 allele have a greater risk of developing dementia at an early age and the prognosis is much worse for those who are APOE epsiln4/epsilon4 homozygote (22). As such, the data was analyzed with the idea of detecting differences of cognitive performance of individuals with or without APOE epsilon4 in each of the vitamins and NSAID subgroups.

A detailed epidemiological study with a large sample size was needed to test this hypothesis, since only a very small percentage of the population consumes both anti-oxidant vitamins and NSAIDs. The Cache County Study in Utah, a longitudinal investigation of more than 5000 community-dwelling participants monitored closely since 1995, offers the unique opportunity to perform such an analysis (7, 23). The experimental results discussed herein focus on analyzing the data from Cache County Study participants who have been taking a combination of NSAIDs and anti-oxidant vitamins (“super-users”).

Apolipoprotein E (APOE) genotype strongly predicts the age of onset, severity, and rate of progression in Alzheimer's disease (20, 21). Individuals with one copy of APOE e4 allele have a greater risk of developing dementia at an earlier age and the prognosis is worse for those who are APOE e4/e4 homozygote (22). As such, the data was analyzed with particular interest in detecting differences in cognitive performance of individuals with or without APOE e4 in each of the vitamins and NSAID subgroups.

Experimental Methods

As discussed above, the Cache County Study on Memory, Health, and Aging is a prospective cohort study of health, cognitive impairment, and dementia in elderly residents of Cache County, Utah. Details of the study design have been published elsewhere (7, 23). Briefly, all residents of the county aged 65 or older as of Jan. 1, 1995, were invited to participate. The institutional review boards of Utah State University, Duke University, and Johns Hopkins University approved all protocols. Informed consent was obtained from all participants; spouses or next of kin gave informed consent when participants were unable to provide it. Buccal DNA samples were voluntarily provided by participants for APOE genotyping.

### Study Sample

A total of 5,092 elderly individuals from Cache County, 90% of those eligible in the county, participated in initial phase of the study. At baseline, the present study identified 356 cases of dementia; these individuals were excluded from the current analyses, leaving a total potential sample of 4736. Another 157 persons did not complete the cognitive tests at baseline and were also excluded. A further requirement for comparison and study herein was that they had at least two cognitive evaluations throughout the eight years of follow-up. Based thereon data from another 1,324 individuals who did not have at least 2 cognitive scores were further excluded as well. Also thirty-six participants (1.1%) had some missing data other than 3MS and were excluded. The remaining 3376 participants who were not demented at baseline and who had cognitive evaluations at multiple time points as well as complete medical data were included in the study. Also, throughout the study, some subjects died and some dropped out for other reasons.

Assessment Procedures

At baseline (1995-1997), participants were evaluated for cognitive function and dementia with a complete multi-stage screening and assessment procedure that included administration of an adapted Modified Mini-Mental State Exam (3MS).(23-27) The 3MS test examines several areas of cognition including orientation to time and place, short term memory, long-term memory, language comprehension, verbal fluency, abstract thinking, and executive function.(25-27) The scores range from 0 to 100. Participants' medical history and family history was obtained with in-depth interviews.

At Wave II (1998-1999), approximately 3 years later (range 2-5 years, standard deviation=0.44), a second evaluation took place (Wave II), using similar procedures in all available surviving participants who had not been diagnosed with dementia at the initial evaluation. At Wave III (2003-2004), approximately 5 years later the 3MS and in-depth interviews were conducted for the surviving participants.

Vitamins and NSAID Use

At the baseline interview and all subsequent interviews, participants were asked to identify all supplements, prescription drugs, and over the counter medications used. This information was supplemented with a visual examination of the items in the participants' medicine chest. Supplement or non-Aspirin NSAID use was coded dichotomously, where users were those who reported regular use, four or more times per week, for a month or more. Vitamin E and C were also coded in the case where a multivitamin was regularly taken that contained a dose similar to those in standard supplements (i.e. 400 International Units of alpha-tocopherol and 500 milligram of vitamin C). The baseline interview also covered topics related to cognition such as socio-economic factors, occupational history, family history of cognitive disturbances and medical history.

Other Risk Factors

Individuals or their informants provided detailed medical histories at the baseline interview. Because of the critical role of vascular risk factors contributing to dementia, participants were queried about hypertension, hypercholesterolemia, diabetes mellitus, stroke, coronary artery bypass graft surgery (CABG), and myocardial infarction (MI). Interviewers recorded a positive history of each condition if the participant (or his/her proxy informant) indicated he/she was ever told (i.e. diagnosed) by a doctor or nurse about the condition, and/or if received treatment for it. A negative history was recorded if the participant did not specify a doctor's diagnosis or treatment.

Statistical Analysis

Demographic comparisons evaluated potential differences in age, sex, education, APOE genotype, and history of cardiovascular factors between individuals with exposure to vitamins E/C and/or NSAIDs. Subjects were subdivided into 5 mutually exclusive groups: 1) Non-users (no vitamin E/C or NSAID use), 2) NSAIDs only users, 3) Vitamin E and C only users, 4) Super Users (Vitamin E/C plus NSAIDs), and 5) Users of vitamin E or C, and NSAIDs). Continuous variables were examined using one-way analysis of variance and categorical variables were examined via χ2 tests. All analyses were performed using SAS version 8.0.

To estimate the differences in trajectory of cognitive decline with regards to vitamins and NSAIDs exposures over time, the data was analyzed with Random effects models (32), using the SAS Proc Mixed procedure (SAS). The Random effects model, also known as Random Regression Model or Random Coefficient Model, is a fairly new form of logistic regression analysis where regression coefficient (beta) is not fixed; rather it is a random variable with particular mean values and standard deviation. Random effect model is particularly well suited for data from repeated outcome measurements from the same individuals over time, such as in this study. The estimated values generated can help to account for individual differences in baseline performance on the 3MS, the correlated nature of successive 3MS scores, and fixed effects of baseline coefficients such as sex, educational attainment, APOE e4 status, and cardiovascular history.

The primary relationship of interest was assessed by fitting interaction terms between the variables for time and the five user groups. Parameterized in this way, the main effect of terms for the user groups provides an estimate of mean differences of SMS scores at baseline between each group and non-users, while the interaction terms provide estimates of the differences in rates of changes of the 3MS over time among the users versus non-users. To assess whether the relationship differed by APOE genotype, the sample was stratified by the presence or absence of an APOE epsilon 4 allele and the random effects model estimated in the full sample, this time including all two-way and three way interaction terms between time, the different user groups, and dichotomized APOE genotype (epsilon 4 positive versus negative).

Also, to examine the effect of these constituents on differential mortality, logistic regression was used to estimate the relative risk of mortality over the course of follow-up among the 5 user groups. These analyses, were controlled for age, sex, education, and APOE genotype status. All analyses were performed using SAS statistical software package version 8.0.

Results

Baseline demographic characteristics of the sample are presented in Table 1. Of the 3376 participants included in the current analyses, 59.4% were non-users, 4,4% were vitamin E and C alone users, 10% were vitamin or vitamin C without NSAID users, and 2.5% were super-users (vitamin E, vitamin C, and NSAIDs). The different user groups were similar to non-users in terms of mean age and education level, bout were more likely to be femal (P less than 0.05). NSAID alone users were also more likely to have hypertension (P less than 0.001) and hypercholesterolemia (P less than 0.05) but less likely to have a stroke (P less than 0.05). The super users were more likely to have hypercholesterolemia (P less than 0.05), and the vitamin E and vitamin C alone users were more likely to be epsilon 4 positive.

Table 2 shows the unadjusted 3MS scores at each wave and changes in scores between Waves I and III for each of the different user groups. Over the 8 years of follow-up, super-users tended to show less decline in 3MS scores (1.37 points, 95% confidence interval [CI] 0.08-2.66) compared to non-users (2.89 points, 95% CI 2.44-3.33). This favorable difference in rate of decline between super-users and non-users was particularly pronounced among those who were epsilon 4 positive. Super users in the epsilon 4 group maintained their level of performance and actually improved over time by a mean of 0.65 points (95% CI-0.58 to 1.89) compared to non-users who declined by a mean of 3.68 points (95% CI 2.93 t 4.44 points). By contrast, the other user groups appeared to decline at the same rate as non-users in the whole sample and among those who were either epsilon 4 positive or negative.

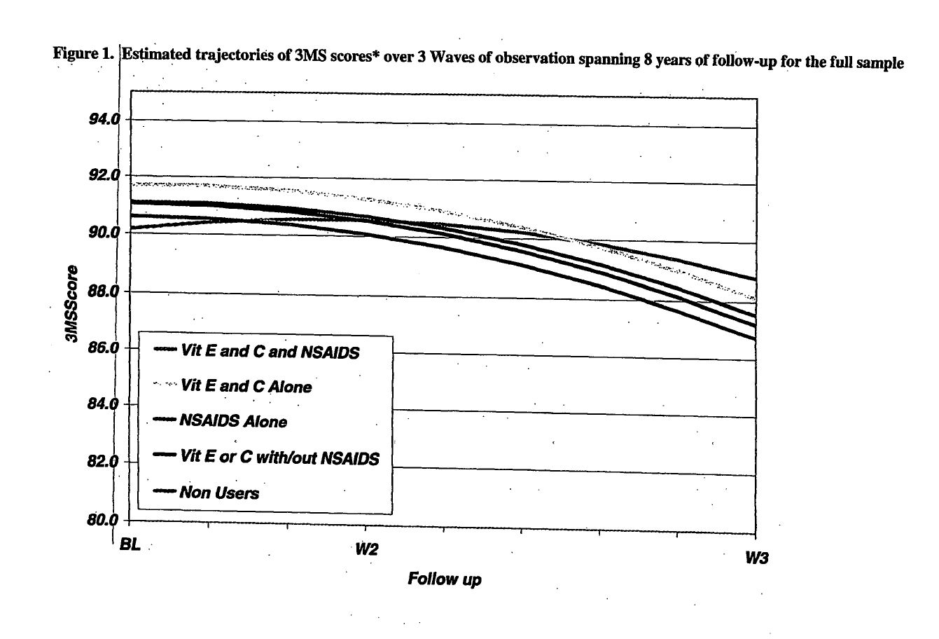

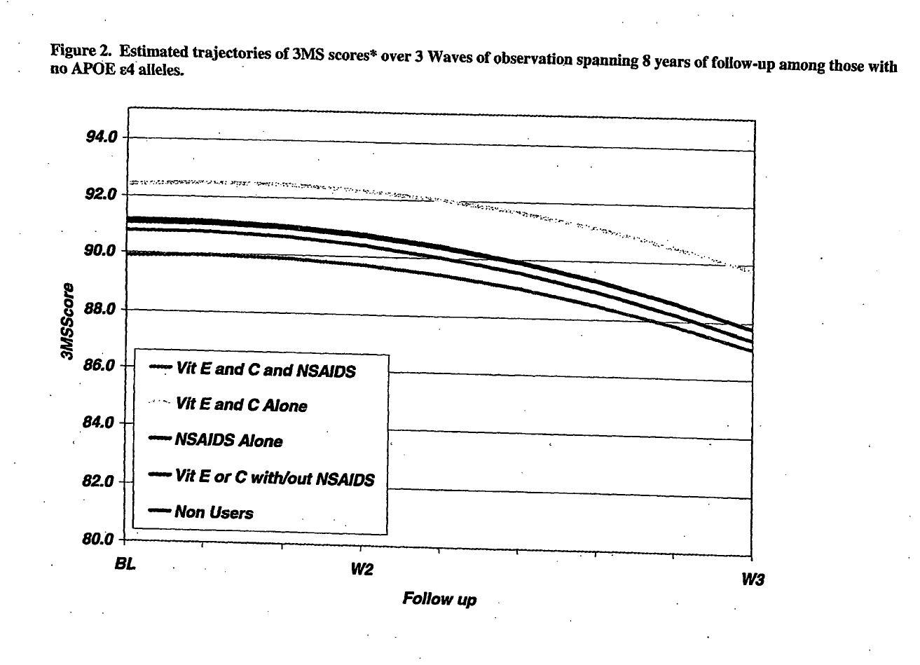

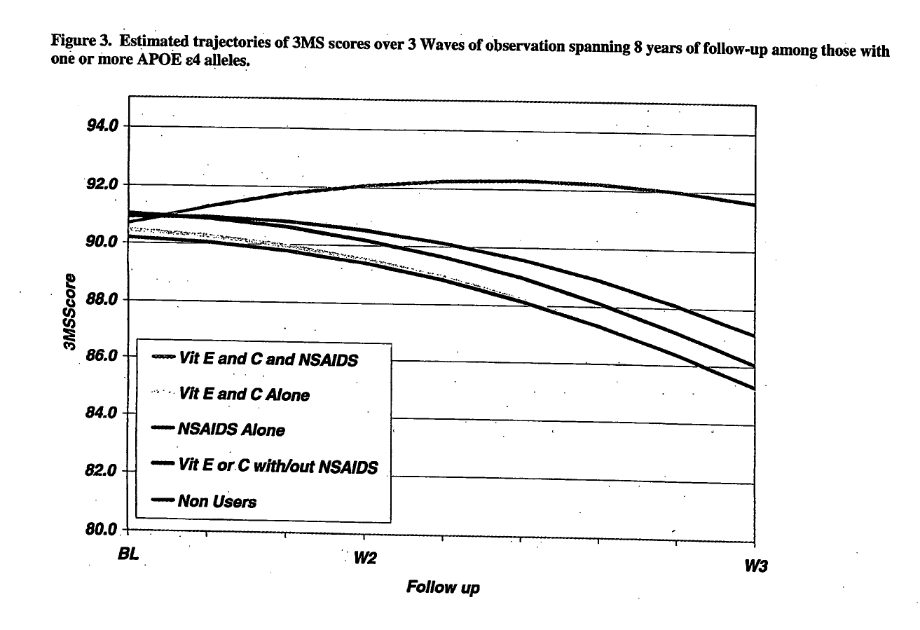

Random effects models were used to examine changes in 3MS scores over time in relation to vitamin and NSAID use while controlling for potential confounders such as age, sex, years of education, history of diabetes, and history of a cardiovascular event, including stroke, coronary artery bypass graft surgery, or myocardial infarction. (Table 3). It was found that users of vitamin E and C performed on average 1.09 points (95% CI 0.28-191) better on the 3MS scale at baseline than non-users. None of the other user groups showed better performance at baseline. Over time, however, only the super-users appeared to do better than the reference group of non-users. It was estimated that super-users declined less than non-users by 0.31 (95% CI 0.04-0.58) points per year, which translates into a difference of about 1 point every 3 years. Similar to what was observed in the unadjusted delta score analysis above, the improved performance over time among super-users was particularly evident among those who were APOE epsilon 4 positive. It was estimated that among APOE epsilon 4 positive participants, super-users declined less than non-users by 0.73 (95% CI 0.29-1.17) points per year, or more than 2 points less every 3 years. All of the other user groups appeared to decline at approximately the same rate as non-users. Among those who were epsilon 4 negative, vitamin E and C users performed better than non-users at baseline by 1.62 points (95% CI 0.59-2.64) and every visit thereafter. However, none of the user groups performed any better than the non-users over time. To formally determine whether the slower rates of decline in super-uses versus non-users was different for those who were epsilon 4 positive compared to epsilon 4 negative, the random effects model was estimated in the full sample and included a three way interaction term between time, the super-user category, and dichotomized APOE genotype. This three way interaction was significant at p less than 0.05. The estimated trajectories of 3MS scores over the 3 waves of evaluation are shown in FIGS. 1-3 for each of the user groups in the whole sample and stratified by the presence or absence of the APOE epsilon 4 allele.

Also, an analysis was effected to assess whether there was an increased risk of mortality among any of the user groups that might potentially bias the observed associations with cognitive performance over time. No difference was observed with regard to risk of mortality in any of the groups, as compared to non-users. The adjusted odds ratio was 1.15 (0.89-1.47) for the NSAIDs alone group, 0.93 (0.52-1.55) for the Vitamin E and C alone group, 1.03 (0.71-1.47) for the vitamin E or C with/out NSAIDs group, and 1.14 (0.55-2.16) for the super-users. There was similarly no evidence of increased mortality for the different user groups compared to non-users among participants who were epsilon 4 positive or negative.

Conclusions:

In this study of 3,376 elderly men and women living in their own community, taking a combination of anti-oxidant vitamins E and C plus NSAIDs was associated with less cognitive decline over time, especially in those individuals with one or more APOE-e4 allele(s). Those taking vitamin E and C, especially if they did not have any APOE-e4 alleles also performed better at baseline, though their trajectory of cognitive decline remained parallel to that of non-users. These results show that there is a superior benefit for using the combination of anti-oxidant vitamins and NSAIDs rather than these constituents alone.

The role of anti-oxidant vitamins with or without NSAIDs in preventing or treating Alzheimer's remains highly controversial. Numerous studies have examined the role of vitamin E (with or without vitamin C) and some, but not all, found a protective effect in slowing cognitive decline or preventing Alzheimer's (4, 16, 19, 28-37). Similarly, more than 25 studies have examined the role of NSAIDs in cognitive function and Alzheimer's disease (5-7, 13, 18). The majority but not all found a protective effect; some did not detect any positive benefit (38-40). The seeming discrepancy among these studies remains puzzling; it may relate to the variations in study populations, duration of usage, dosages of agents, or inclusion/exclusion criteria in these studies. Also, the disparate results may be explained by the complexity of the processes leading to brain atrophy for example in Alzheimer's disease and also in the wide range of effects elicited by anti-oxidant vitamins and NSAIDs and the possible effects thereof on these processes.

According to the current amyloid hypothesis of Alzheimer's disease (1, 3, 6), when amyloid aggregates into insoluble plaques, a significant inflammatory response is triggered which leads to neurodegeneration. The cascade of inflammatory processes follows 2 main directions in parallel; one involves production of free radical ROS while the other involves the release of cytokines, prostaglandins, proteases, chemokines, and complement proteins by activated microglia in the brain. Microglia release cytokines, prostaglandins, proteases, chemokines, complement proteins, and reactive oxygen species (ROS). To stop these processes, NSAIDs possibly reduce and inhibit prostaglandins, but would have no effect on ROS. Similarly, anti-oxidant vitamins can lower and minimize the tissue injury caused by ROS, but would have no effect on cytokines or complement proteins. Given the large number of molecules and steps involved in the pathophysiology of Alzheimer's disease, it is possible that more than one agent, as found in the present invention, would be necessary to halt or slow the cascade of reactions. In fact, Alzheimer's may be similar to other complex diseases such as AIDS, cardiac disease, or tuberculosis where multiple medications are needed for, prevention or treatment. The fact that more benefit was noted for super-users who were positive for APOE-e4 may be due to the presence of more inflammation in their brains, and thus a richer substrate and a better opportunity for a multi-prong intervention.

Yet another explanation for synergistic benefit of anti-oxidant vitamins and NSAIDs in slowing cognitive decline may be their roles in mechanisms above and beyond inflammation. and amyloid formation Cerebral disease, formation of axonal tangles (with phosphorylated tau), NMDA toxicity, have also been implicated in Alzheimer's neurodegeneration.(41). Vitamin E can have a role in lowering the impact of arteriosclerosis in the brain, since it can inhibit smooth cell proliferation in blood vessels, diminish oxidization of LDL, and reduce platelet aggregation (4, 42). NSAIDs can reduce NMDA toxicity, inhibit gamma-secretase, and slow amyloid plaque aggregation (45). Thus, vitamins E/C and NSAIDs can complement each other in lowering the impact of brain pathology in dementia in areas beyond prostaglandins and reactive oxygen species. Alzheimer's may be similar to other complex diseases such as AIDS, asthma, heart failure or tuberculosis where multiple medications are needed for prevention and/or treatment. The fact that more benefit was seen in super-users who were epsilon 4 positive may be due to the presence of more pathology in their brains, and thus a richer substrate and a better opportunity for a multi-prong intervention.

Controversy over the superior benefit of natural (dietary) vs manufactured sources of vitamin E on cognition continues Dietary sources of vitamin E are reportedly effective in maintaining cognitive function and lowering the risk of Alzheimer's especially in low-risk individuals who lack APOE epsilon 4 alleles. (16, 29, 34-36). Interestingly, this may also involve the parallel processes of ROS and inflammation. While dietary vitamin E contains all 4 forms of tocopherols (alpha, beta, delta, and gamma), supplements contain mainly alpha-tocopherol. (16, 29, 34-36) The alpha-tocopherol component has strong anti-oxidant properties while the gamma-tocopherol component has strong anti-inflammatory properties. (46,47) Thus dietary sources offer components that can both absorb ROS molecules and minimize inflammation. Adding vitamin C to vitamin E helps to recycle vitamin E from its oxidized form to a reduced state where it can serve to lower ROS again and again. By regenerating the vitamin E, vitamin C brings it to a level that can be as effective as natural vitamin E and all its different forms. More studies are need to shed light on the variability of finding in studies reporting dietary sources of vitamin E (or C) and their supplement variants.

The studies discussed supra relied on participants' recall to obtain information about their use of medications and supplements. In individuals at risk for developing dementia, this may cause a bias. However, to take this into account their medicine chest inventory was further assessed and information was procured from spouses or next of kin when there was a question of dementia. Another limitation was some loss of participants to follow-up due to reasons such as death or location out of the area. Fortunately, there was more than 80% follow-up for each phase of the study. A third limitation was basing the conclusions on the use of drugs by participants from only their first visit (and not allowing for the fact that some may change their pattern of medication or supplement use). Prior experience with this group has indicated that those who take supplements continue taking them and very few individuals discontinue or change them (18). However, there were some new users of vitamin supplements at Wave II. These individuals were excluded them and the data re-analyzed. Since the number of new users was small, their exclusion did not make a statistical difference on the final results (data not shown).

The strength of the study included the large sample size of more than 3000 participants. The original sample comprised more than 90% of the elderly in the community, and there was little question of biased selection. These findings cannot be explained by “healthy-user” bias alone. Individuals with healthy life-style were already taking vitamin E, C, or a multi-vitamin supplement. If these results were due to a healthy-user bias, it would have been expected to see a significant protective effect among users of vitamin E or C alone; but this was not the case.

Recent reports have raised concerns about the potential risk of increased mortality in some elderly patients taking high doses of vitamin E (48, 49). In the study, no significant difference in mortality in users of anti-oxidant vitamins and/or NSAIDs was observed as compared to non-users. A recent placebo-controlled clinical trial that examined the role of vitamin E and donepezil in slowing the rate of cognitive decline in the elderly also did not note any increased mortality associated with using vitamin E (50). Moreover, in the Women's Health Study, involving 39,876 healthy participants monitored for over 10 years, there was no significant difference in mortality between those taking vitamin E and placebo (51). In fact, there was a trend toward decreased cardiovascular mortality in healthy women using vitamin E. Preliminary data from subgroup analysis in Cache County suggests that vitamin E may only be harmful among elderly with prior cardiovascular disease, and not in otherwise healthy participants.

A main strength of the study herein is the large sample size of more than 3000 participants monitored closely over 8 years. The original sample comprised more than 90% of the elderly in the community, and as a result there was less concern for selection bias. Furthermore, it is unlikely that the findings can be explained by “healthy bias” alone. Individuals with healthy life styles were already taking vitamin E, C, or a multi-vitamin supplement. If the results were due to healthy user bias, one would expect to see a significant effect among users of vitamin C and E alone, but this was not the case. Moreover, taking NSAIDs is not considered as a means of successful aging, and users had medical conditions requiring such treatment.

It was also found that super-users who had one or more APOE-e4 alleles gained more benefit from the combined use of vitamins E and C plus NSAIDs than those without any APOE-e4 alleles. This finding suggests that taking the combination of these compounds may be of most benefit to those already at heightened risk of developing dementia. Perhaps these individuals, as a group, are more likely in the indolent phase of dementia, have more inflammation in their brain (52), and hence are more inclined to show improvement on the 3MS score when compared to super-users without any APOE-e4 alleles. APOE epsilon 4 negative participants most likely do not have the same degree of inflammatory reactions in their brains, and thus less likely to benefit from concentrated multi-prong anti-inflammatory intervention.

These results suggest that a cocktail of vitamin E/C and NSAIDs can be considered for primary prevention of dementia in individuals with memory concerns and a positive APOE-e4 polymorphism or those with mild cognitive impairment. A major advantage of a combination therapy would be the opportunity to intervene with the pathogenesis of dementia with multiple compounds at lower doses, thus gaining the most benefit while avoiding the side-effects associated with high doses of the individual ingredients. Moreover, the risk of myocardial infarction with NSAIDs do not apply equally to all members of this class (53) just as not all NSAIDs can inhibit gamma-secretase equally or cross the blood brain barrier (54). For example, low dose ibuprofen can lower amyloid levels, inhibit amyloid aggregation, cross the blood brain barrier, and reduce both lipid peroxidation and ROS while causing minimal cardiovascular risks (43, 44, 55, 56).

In conclusion, the observed superior performance in cognitive tests in elderly using a combination of anti-oxidant vitamins E and C plus NSAIDs, and especially in those with APOE-e4 polymorphism, suggests the use thereof in the prevention of MCI and Alzheimer's disease. These agents are also fairly inexpensive and have been in use among the general public for decades. These constituents should be administered at optimal doses as described herein, since they can have significant side-effects at higher doses. At such dosages this combination provides a novel synergistic combination capable of delaying the onset of cognitive decline in those at risk especially the elderly at high risk for Alzheimer's disease such as APOE epsilon-4 genetic carriers.

All references cited herein are incorporated by reference in their entireties.

### REFERENCES

1. Cummings J L, Cole G. Alzheimer disease. JAMA. May 8, 2002; 287(18):2335-2338.
2. DeKovsky Pathology and pathways of Alzheimer's disease with an update on new developments in treatment. J Am Geriatric Soc 2003:51 (5 Suppl Dementia) S314-320
3. Selkoe D J. The origins of Alzheimer disease: a is for amyloid. JAMA. Mar. 22-29 2000; 283(12):1615-1617.
4. Cole G M, Morihara T, Lim G P, Yang F, Begum A, Frautschy S A. NSAID and Antioxidant Prevention of Alzheimer's Disease: Lessons from In Vitro and Animal Models. Ann N Y Acad Sci. December 2004; 1035:68-84.
5. Zandi P P, Breitner J C. Do NSAIDs prevent Alzheimer's disease? And, if so, why? The epidemiological evidence. Neurobiol Aging. November-December 2001; 22(6):811-817.
6. McGeer P L, McGeer E G. Inflammation and the degenerative diseases of aging. Ann N Y Acad Sci. December 2004; 1035:104-116.
7. Breitner J C, Zandi P P. Do non-steroidal anti-inflammatory drugs reduce the risk of Alzheimer's disease? N Engl J. Med. Nov. 22 2001; 345(21):1567-1568.
8. Yaffe et al., Neurology 2003:61 (1):76-80
9. Engelhardt et al., Arch Neurol. 2004: 61(5):668-672
10. Smith et al., Proc Natl Acad Sci USA 1991 88(23):10540-10543
11. Szekly et al., Neuroepidemiology 2004: 23(4):159-169
12. Townsend et al., FASEB J 2005 19(12):1592-1601
13. Akiyama H, Barger S, Barnum S, et al. Inflammation and Alzheimer's disease. Neurobiol Aging. May-June 2000; 21(3):383-421.
14. Int'l Veld B A et al., New Engl J. Med. 2001 345(21):1515-1521
15. Dik et al., Neurology 2005 64(8):1371-1377
16. Engelhardt et al., JAMA 2002 287(24):3223-3229
17. Smith C D, Carney J M, Starke-Reed P E, et al. Excess brain protein oxidation and enzyme dysfunction in normal aging and in Alzheimer disease. Proc Natl Acad Sci USA. Dec. 1 1991; 88(23):10540-10543.
18. Zandi et al., Neurology 2002 59(6):880-886
19. Zandi et al., Arch Neurol. 2004 61(1):82-88
20. Huang Yl. Neurology 2005, “Apolipoprotein E and Alzheimer's disease”
21. Martins et al. Neurology 2005 65(12):1888-1893
22. Corder et al., Science 1993 261(5123):921-923
23. Miech et al., Neurology 2002 58(2):209-218
24. Breitner et al., Neurology 1999 53(2):321-331
25. Tschanz J T, Welsh-Bohmer K A, Plassman B L, Norton M C, Wyse B W, Breitner J C. An adaptation of the modified mini-mental state examination: analysis of demographic influences and normative data: the cache county study. Neuropsychiatry Neuropsychol Behav Neurol. March 2002; 15(1):28-38.
26. Teng E L, Chui H C. The Modified Mini-Mental State (3MS) examination. J Clin Psychiatry. August 1987; 48(8):314-318.
27. Folstein M F, Folstein S E, McHugh P R. “Mini-mental state”. A practical method for grading the cognitive state of patients for the clinician. J Psychiatr Res. November 1975; 12(3):189-198.
28. Cherubini A, Martin A, Andres-Lacueva C, et al. Vitamin E levels, cognitive impairment and dementia in older persons: the InCHIANTI study. Neurobiol Aging. July 2005; 26(7):987-994.
29. Foley D J, White L R. Dietary intake of antioxidants and risk of Alzheimer disease: food for thought. JAMA. Jun. 26 2002; 287(24):3261-3263.
30. Gray S L, Hanlon J T, Landerman L R, Artz M, Schmader K E, Fillenbaum G G. Is antioxidant use protective of cognitive function in the community-dwelling elderly? Am J Geriatr Pharmacother. September 2003; 1(1):3-10.
31. Grodstein F, Chen J, Willett W C. High-dose antioxidant supplements and cognitive function in community-dwelling elderly women. Am J Clin Nutr. April 2003; 77(4):975-984.
32. Masaki K H, Losonczy K G, Izmirlian G, et al. Association of vitamin E and C supplement use with cognitive function and dementia in elderly men. Neurology. Mar. 28, 2000 2000; 54(6):1265-1272.
33. Mecocci P. Oxidative stress in mild cognitive impairment and Alzheimer disease: a continuum. J Alzheimer's Dis. April 2004; 6(2):159-163.
34. Morris M C, Evans D A, Bienias J L, et al. Dietary intake of antioxidant nutrients and the risk of incident Alzheimer disease in a biracial community study. Jama. Jun. 26 2002; 287(24):3230-3237.
35. Morris M C, Evans D A, Bienias J L, Tangney C C, Wilson R S. Vitamin E and cognitive decline in older persons. Arch Neurol. July 2002; 59(7):1125-1132.
36. Morris M C, Evans D A, Tangney C C, et al. Relation of the tocopherol forms to incident Alzheimer disease and to cognitive change. Am J Clin Nutr. February 2005; 81(2):508-514.
37. Sano M, Ernesto C, Thomas R G, et al. A Controlled Trial of Seligiline, Alpha-Tocopherol, or Both as Treatment for Alzheimer's Disease. N Engl J Med. Apr. 24, 1997 1997; 336(17):1216-1222.
38. Yaffe et al., Neurology 2004 63(9): 1705-1707
39. Luchsinger at el., Arch Neurol 2003 60(2): 203-208
40. de Craen et al., Am J Epidemiol 2005 161(2):114-120
41. Casserly I, Topol E. Convergence of arteriosclerosis and Alzheimer's disease: inflammation, cholesterol, and misfolded proteins. Lancet. Apr. 3 2004; 363(9415):1139-1146.
42. Frautschy S A, Hu W, Kim P, et al. Phenolic anti-inflammatory antioxidant reversal of Abeta-induced cognitive deficits and neuropathology. Neurobiol Aging. November-December 2001; 22(6):993-1005.
43 Weggen et al., J Biol. Chem. 2003 278(34):31831-31837
44. Weggen et al., Nature 20001 414(2660):212-216
45. Eriksen J L, Sagi S A, Smith T E, et al. NSAIDs and enantiomers of flurbiprofen target gamma-secretase and lower Abeta 42 in vivo. J Clin Invest. August 2003; 112(3):440-449.
46. Jiang et al., Free Radical Biol Med 2002 33(11):1534-1542
47. Christen et al., J Lipid Res 2002 43(11):1978-1985
48. Loan E, Bosch J, Yusuf S, et al. Effects of long-term vitamin E supplementation on cardiovascular events and cancer: a randomized controlled trial. Jama. Mar. 16 2005; 293(11):1338-1347.
49. Miller E R, 3rd, Pastor-Barriuso R, Dalal D, Riemersma R A, Appel L J, Guallat E. Meta-analysis: high-dosage vitamin E supplementation may increase all-cause mortality. Ann Intern Med. Jan. 4, 2005; 142(1):37-46.
50. Peterson et al., N Engl J Med. 2005 352(23):2379-2388
51. Lee I M, Cook N R, Gaziano J M, et al. Vitamin E in the primary prevention of cardiovascular disease and cancer: the Women's Health Study: a randomized controlled trial. Jama. Jul. 6 2005; 294(1):56-65.
52. Ophir et al., Neurobiol. Dis 2005 20(3):709-718
53. Garcia et al., BMC Med 2005 3:17
54. Gasparini et al., J Neurochem 2004 91(3); 521-526
55. Dokmeci Folia Med (Ploviiv) 2004 46(2):5-10
56. Thomas et al., Neuroreport 2001 12(15):3263-3267

TABLE 1
Demographic characteristics of 3,376 participants in this Cache County Study at the baseline (Wave I) visit

| | | | | | | | Vit E or C | | Vit E and C | |
|---|---|---|---|---|---|---|---|---|---|---|
| | Non-Users | | NSAIDs alone | | Vit E and C alone | | with/out NSAIDs | | with NSAIDs | |
| Characteristic | (n = 2007) | | (n = 794) | | (n = 153) | | (n = 337) | | (n = 85) | |
| Age, mean (SD), y | 74.3 | (6.6) | 73.9 | (6.3) | 73.6 | (5.5) | 73.4 | (6.1) | 72.9 | (6.1) |
| Female | 1068 | (53.2) | 542 | (68.3) † | 79 | (51.6) ‡ | 229 | (68.0) † | 56 | (65.9) ‡ |
| Education, mean (SD), y | 13.4 | (2.9) | 13.2 | (2.7) | 13.8 | (2.9) | 13.5 | (2.8) | 13.8 | (2.6) |

Cardiovascular risk factors

| Hypertension | 758 | (37.8) | 381 | (48.0) † | 55 | (36.2) | 118 | (35.0) | 39 | (46.4) |
|---|---|---|---|---|---|---|---|---|---|---|
| Myocardial infarction | 221 | (11.1) | 93 | (11.8) | 15 | (10.0) | 39 | (11.7) | * | |
| Stroke | 68 | (3.4) | 24 | (3.1) ‡ | * | | 14 | (4.2) | * | |
| Diabetes | 201 | (10.0) | 99 | (12.5) | * | | 33 | (9.8) | * | |
| Hypercholesterolemia | 337 | (16.9) | 182 | (23.1) ‡ | 28 | (18.4) | 71 | (21.1) | 26 | (30.6) ‡ |

| | | | | | | | | |
|---|---|---|---|---|---|---|---|---|
| CABG | 127 | (6.3) | 48 | (6.1) | * | 21 | (6.3) | * |

Number of APOE ε4 alleles

| | | | | | | | | | | |
|---|---|---|---|---|---|---|---|---|---|---|
| 0 | 1390 | (69.8) | 554 | (70.1) | 93 | (60.8) | 228 | (68.1) | 56 | (65.9) |
| 1 or more | 601 | (30.2) | 236 | (29.9) | 60 | (39.2) ‡ | 107 | (31.9) | 29 | (34.1) |

Abbreviations: SD, standard deviation; CABG, coronary artery bypass graft surgery; APOE, apolipoprotein E gene; NSAIDs, non-steroidal anti-inflammatory medication.
* Values suppressed in to comply with the Center for Medicare and Medicaid Services privacy guidelines
† p < .001; comparisons are made to reference group of non-users
‡ p < .05; comparisons are made to reference group of non-users
Data are expressed as number (percentage) unless otherwise specified.

TABLE 2
Mean 3MS scores and 95% confidence intervals for 3,376* Cache County residents over 3 Waves of observation spanning
8 years of follow-up for the full sample and stratified by the presence or absence of an APOE ε4 allele

| | | | | | | | |
|---|---|---|---|---|---|---|---|
| | | Wave I | | Wave II | | Wave III | Mean Change |
| Group | n | Mean (95% CI) | n | Mean (95% CI) | n | Mean (95% CI) | Wave I to III** |

Full Sample

| | | | | | | | | | | | |
|---|---|---|---|---|---|---|---|---|---|---|---|
| Non-Users | 2006 | 90.93 | (90.68-91.17) | 1954 | 90.31 | (89.94-90.68) | 1304 | 89.0 | (88.48-89.51) | −2.89 | (−3.33-−2.44) |
| NSAIDs alone | 794 | 91.68 | (91.33-92.03) | 770 | 91.45 | (90.94-91.96) | 539 | 89.34 | (88.58-90.10) | −3.05 | (−3.74-−2.37) |
| Vit E and C alone | 153 | 92.16 | (91.41-92.90) | 151 | 91.85 | (90.86-92.84) | 110 | 88.96 | (87.35-90.57) | −3.52 | (−5.00-−2.04) |
| Vit E or C with/out | 337 | 91.80 | (91.22-92.37) | 330 | 91.37 | (90.62-92.12) | 218 | 89.58 | (88.42-90.74) | −3.47 | (−4.52-−2.41) |
| NSAIDs | | | | | | | | | | | |
| Vit B and C with | 85 | 90.93 | (89.80-92.06) | 82 | 91.98 | (90.63-93.32) | 59 | 90.56 | (89.13-91.99) | −1.37 | (−2.66-−0.08) |
| NSAIDs | | | | | | | | | | | |

0 APOE ε4 alleles

| | | | | | | | | | | | |
|---|---|---|---|---|---|---|---|---|---|---|---|
| Non-Users | 1389 | 91.05 | (90.76-91.34) | 1359 | 90.52 | (90.08-90.95) | 902 | 89.44 | (88.82-90.06) | −2.54 | (−3.09-−1.98) |
| NSAIDs alone | 554 | 91.63 | (91.20-92.07) | 538 | 91.57 | (90.97-92.16) | 370 | 89.37 | (88.45-90.29) | −2.95 | (−3.78-−2.12) |
| Vit E and C alone | 93 | 92.90 | (92.02-93.79) | 91 | 92.98 | (91.81-94.14) | 70 | 90.73 | (89.24-92.22) | −2.76 | (−4.13-−1.39) |
| Vit E or C with/out | 228 | 91.72 | (90.99-92.45) | 225 | 91.33 | (90.45-92.22) | 144 | 89.97 | (88.65-91.29) | −3.08 | (−4.33-−1.84) |
| NSAIDs | | | | | | | | | | | |
| Vit E and C with | 56 | 90.48 | (88.98-91.98) | 54 | 90.69 | (88.85-92.52) | 36 | 89.0 | (86.94-91.06) | −2.67 | (−4.56-−0.78) |
| NSAIDs | | | | | | | | | | | |

1 + APOE ε4 alleles

| | | | | | | | | | | | |
|---|---|---|---|---|---|---|---|---|---|---|---|
| Non-Users | 601 | 90.63 | (90.16-91.10) | 579 | 89.79 | (89.07-90.52) | 398 | 87.96 | (87.04-88.89) | −3.68 | (−4.44-−2.93) |
| NSAIDs alone | 236 | 91.82 | (91.20-92.43) | 230 | 91.29 | (90.30-92.27) | 166 | 89.55 | (88.22-90.88) | −3.02 | (−4.19-−1.84) |
| Vit E and C alone | 60 | 91.00 | (89.70-92.30) | 60 | 90.13 | (88.43-91.83) | 40 | 85.88 | (82.40-89.35) | −4.85 | (−8.21-−1.49) |
| Vit E or C with/out | 107 | 92.02 | (91.11-92.93) | 103 | 91.54 | (90.11-92.98) | 73 | 88.88 | (86.56-91.20) | −4.12 | (−6.12-−2.12) |
| NSAIDs | | | | | | | | | | | |
| Vit E and C with | 29 | 91.79 | (90.08-93.50) | 28 | 94.46 | (93.0-95.93) | 23 | 93.00 | (91.66-94.34) | 0.65 | (−1.89-0.58)*** |
| NSAIDs | | | | | | | | | | | |

*Of the 3,376 participants included in the analysis, 2,140 provided 3MS scores at all three waves and 1,236 provided 3MS scores at two of the three waves.
**Mean change was calculated by taking the mean of changes in 3MS scores from Wave III minus Wave I for those participants who had both.
***Mean scores within each Wave and mean change scores for each user group were compared against the reference group of non-users with t-tests in the full sample and in sub-groups formed by the presence or absence of the APOE ε4 allele; differences at p < 0.01 are shown.

TABLE 3
Mixed models of 3MS scores for 3,376 study participants over 3 Waves of observation spanning 8 years
of follow-up for the full sample and stratified by the presence or absence of an APOE ε4 allele

| Unadjusted | | Adjusted* | |
|---|---|---|---|
| β (95% CI)** | β × time (95% CI) | β (95% CI) | β × time (95% CI) |

Full Sample

| Non-Users | Reference | | | | Reference | | | |
|---|---|---|---|---|---|---|---|---|
| NSAIDs alone | 0.79 | (0.35-1.23) | 0.04 | (−0.06-0.15) | 0.49 | (0.08-0.90) | 0.04 | (−0.06-0.14) |
| Vit E and C alone | 1.26 | (0.39-2.14) | 0.05 | (−0.16-0.25) | 1.09 | (0.28-1.91)*** | 0.04 | (−0.17-0.24) |
| Vit E or C with/out | 0.91 | (0.29-1.52) | −0.01 | (−0.15-0.14) | 0.44 | (−0.13-1.02) | 0.004 | (−0.14-0.15) |
| NSAIDs | | | | | | | | |
| Vit E and C with | 0.13 | (−1.03-1.29) | 0.32 | (0.05-0.60) | −0.44 | (−1.52-0.64) | 0.31 | (0.04-0.58)*** |
| NSAIDs | | | | | | | | |

0 APOE ε4 alleles

| Non-Users | Reference | | | | Reference | | | |
|---|---|---|---|---|---|---|---|---|
| NSAIDs alone | 0.65 | (0.13-1.17) | 0.01 | (−0.11-0.14) | 0.40 | (−0.09-0.89) | −0.001 | (−0.12-0.12) |
| Vit E and C alone | 1.90 | (0.79-3.01) | 0.12 | (−0.14-0.37) | 1.62 | (0.59-2.64)*** | 0.10 | (−0.16-0.35) |
| Vit E or C with/out | 0.69 | (−0.05-1.44) | 0.008 | (−0.17-0.19) | 0.28 | (−0.41-0.97) | 0.01 | (−0.16-0.19) |
| NSAIDs | | | | | | | | |
| Vit E and C with | −0.46 | (−1.88-0.95) | 0.09 | (−0.25-0.44) | −0.90 | (−2.22-0.43) | 0.07 | (−0.27-0.41) |
| NSAIDs | | | | | | | | |

1 + APOE ε4 alleles

| Non-Users | Reference | | | | Reference | | | |
|---|---|---|---|---|---|---|---|---|
| NSAIDs alone | 1.17 | (0.36-1.98) | 0.15 | (−0.04-0.34) | 0.73 | (−0.03-1.49) | 0.14 | (−0.05-0.33) |
| Vit E and C alone | 0.38 | (−1.04-1.81) | −0.03 | (−037-0.31) | 0.25 | (−1.10-1.60) | −0.04 | (−0.37-0.30) |
| Vit E or C with/out | 1.44 | (0.33-2.55) | −0.001 | (−0.26-0.26) | 0.83 | (−0.21-1.87) | −0.01 | (−0.26-0.25) |
| NSAIDs | | | | | | | | |
| Vit E and C with | 1.34 | (−0.67-3.34) | 0.75 | (0.30-1.20) | 0.49 | (−1.38-2.36) | 0.73 | (0.29-1.17)*** |
| NSAIDs | | | | | | | | |

*Adjusted for baseline age, sex, education, history of diabetes, history of a cardiovascular event, and in the full sample APOE status (1 or more ε4 alleles vs none)
**Parameters from the mixed models; β estimates the mean difference in 3MS scores at baseline between each user group and the reference group of non-users; β × time estimates the difference in rate of decline on 3MS scores per year between each user group and the reference group of non-users

As will be apparent to those skilled in the art in light of the foregoing disclosure, many alterations and modifications are possible in the practice of this invention without departing from the spirit or scope thereof. Accordingly, the scope of the invention is to be construed in accordance with the substance defined b_{y} the following claims.

1. A medicament or health supplement composition for preventing and/or delaying the onset or progression of dementia in individuals at elevated risk thereof which comprises synergistically effective amounts of the following:
(i) vitamin C (calcium ascorbate):
(ii) vitamin E (d-alpha tocopherol):
(iii) DHA (desosahexaenoic acid); and
(iv) at least one NSAID (non-steroidal anti-inflammatory drug).

2. The medicament or health supplement of claim 1 wherein the at least one NSAID comprises t least one of ibuprofen, suldinac, meclofenamic acid, aspirin, diclofenac sodium, naproxen, flurbiprofen, nabumetone, acetylsalicyclic acid, diflunisal, choline magnesium trisalicyclate, benorylate, flufenamic acid, mefanamic acid, niflumic acid, fenclofenac, alclofenac, fentiazac, ketoprofen, fenoprofen, fenbufen, suprofen, indoprofen, tiaprofenac acid, benoxaprofen, piroprofen, tolmetin, zomepirac, clopinac, indomethacin, phenylbutazone, oxyphenbutazone, azapropazone, feprazone, piroxicam, isoxicam and sudoxicam.

3. The medicament of heath supplement of claim 2 wherein the NSAID comprises ibuprofen.

4. The medicament or health supplement of claim 1 which is suitable for once-daily administration.

5. The medicament of claim 1 which is suitable for twice-daily administration.

6. The medicament of claim 1 which comprises not more than 3000 mg of vitamin C, not more than 1000 iu of vitamin E, not more than 3000 mg of DHA, and at least one NSAID wherein the amount thereof is not more than 400 mg if ibuprofen, not more than 300 mg if suldinac, not more than 150 mg if meclofenamic acid, not more than 150 mg if aspirin, not more than 150 mg if diclofenac sodium, not more than 500 mg if naproxen, not more than 300 mg of flurbiprofen and not more than 1000 mg if nabumetone.

7. The medicament or health supplement of claim 1 wherein the amount of vitamin C ranges from 100-2000 mg, the amount of vitamin E ranges from 50-600 iu, the amount of DHA ranges from 100-2000 mg, and the amount of the at least one NSAID ranges from 20-200 mg if ibuprofen, 10-200 mg if suldinac, 5-100 mg if meclofenamic acid, 10-100 mg if aspirin, 5-100 mg if diclofenac sodium, 20-200 mg if naproxen, 20-200 mg if flurbiprofen, and 50-500 mg if nabumetone.

8. The medicament or health supplement of claim 1 wherein the amount of vitamin C ranges from 20-1000 mg, the amount of vitamin E ranges from 100-400 iu, the amount of DHA ranges from 200-800 mg, and the amount of said at least one NSAID ranges from 30-100 mg if ibuprofen, 30-100 mg if suldinac, 10-40 mg if meclofenamic acid, 20-50 mg if aspirin, 10-40 mg if diclofenac sodium, 50-150 mg if naproxen, 30-100 mg if flurbiprofen, and 100-300 mg if nabumetone.

9. The medicament or health supplement of claim 1 wherein the amount of vitamin C is 500 mg, the amount of vitamin E is 200 iu, the amount of DHA This 50 mg, and the amount of the at least one NSAID is 50 mg if ibuprofen, 50 mg if suldinac, 20 mg if meclofenamic acid, 30 mg if aspirin, 20 mg if diclofenac sodium, 80 mg if naproxen, 50 mg if flurbiprofen, and 150 mg if nabumetone.

10. The medicament or health supplement which comprises a tablet formulation.

11. The medicament or health supplement of claim 1 which comprises a capsule formulation.

12. The medicament or health supplement of claim 1 which comprises at least one coating.

13. The medicament or health supplement of claim 12 wherein the coating is an enteric coating.

14. A method of preventing and/or delaying the onset or progression of dementia in an individual at risk thereof which comprises administering an effective amount of a medicament according to any one of claims 1-13.

15. The method of claim 14 wherein the medicament or health supplement is administered once-daily.

16. The method of claim 14 medicament or health supplement is administered twice-daily.

17. The method of claim 14 wherein the individual is at elevated risk of developing dementia because of advanced age, family history, environmental factors, and/or a genotype that correlates to increased risk of dementia.

18. The method of claim 14 wherein the individual has a genotype correlated to an elevated risk of Alzheimer's disease or a family history of Alzheimer's disease.

19. The method of claim 14 wherein the individual has an APOE allele characteristic of increased incidence of Alzheimer's disease.

20. The method of claim 19 wherein the individual is an APOE epsilon-4 carrier.
